(19) United States
(12) Patent Application Publication
(10) Pub. No.: US 2006/0088571 A1
(43) Pub. Date: Apr. 27, 2006

(54) Biocompatible and hemocompatible polymer compositions

(75) Inventors: Mingfei Chen, Santa Rosa, CA (US); Kishore Udipi, Santa Rosa, CA (US); Peiwen Cheng, Santa Rosa, CA (US); Ron Sundar, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/970,171

(22) Filed: Oct. 21, 2004

(51) Int. Cl. C08F 120/10; A61F 2/00

(52) U.S. Cl. 424/426; 525/330.3; 604/500

(57) ABSTRACT

Biocompatible coatings for medical devices are disclosed. Specifically, polymer coatings designed to control the release of bioactive agents from medical devices in vivo are disclosed. The present application also discloses providing vascular stents with controlled release coatings and related methods for making these coatings. Additional embodiments of the present invention include stents coated with the disclosed copolymer(s) and peptide drugs. Methods for treating or inhibiting post-stent implantation restenosis in patients are also provided.

### FIELD OF THE INVENTION

This invention relates generally to biocompatible and hemocompatible coatings for medical devices. More specifically, the present invention relates to polymer coatings designed to control the release of peptides from a medical device. Even more specifically the present invention relates to providing vascular implants with controlled release coatings and related methods for making these coatings.

### BACKGROUND OF THE INVENTION

Medical devices are used for myriad purposes on and throughout an animal's body. They can be simple ex vivo devices such as adhesive bandages, canes, walkers and contact lenses or complex implantable devices including pacemakers, heart valves, vascular stents, catheters and vascular grafts. Implantable medical devices must be biocompatible to prevent inducing life threatening adverse physiological responses between the implant recipient and device.

Recently, highly biocompatible polymers have been formulated to provide implantable medical devices with coatings. These coating not only increase an implant's tissue compatibility but can also function as bioactive agent reservoirs. However, designing polymer coatings for medical devices has proven problematic. All medical device coatings must be non-toxic, durable and adhere well to device surfaces. Additionally, when the medical device comes into intimate contact with unprotected tissues such as blood and internal organs it must also be biocompatible. Furthermore, if the medical device is designed to be pliable either in operation or deployment, the coating must resist cracking, fracture and delamination.

Moreover, medical devices intended to act as bioactive agent (drug) reservoirs must not only be biocompatible, structurally stable and resistant to delamination, but also chemically compatible with the drug to be deployed. Furthermore, if the reservoir is also intended to control the drug's release rate into adjacent tissue the polymer used must possess other highly specialized properties as well.

Drug-polymer physical chemistry and the physical characteristics of the coating itself, such as coating thickness, are the two most important factors in determining a polymer matrix's drug elution profile. Highly compatible drug-polymer combinations usually result in more even elution rates and are therefore preferable for most in vivo applications. Polymer-drug compatibility is a function of drug-polymer miscibility. The degree of miscibility, or compatibility, between a drug and a polymer carrier can be ascertained by comparing their relative solubility parameters. However, as will be more fully developed below, balancing drug elution rates with biocompatibility, ductility and adhesiveness requires more than merely matching a single polymer with a drug based on their total solubility parameters alone.

Cardiovascular disease, specifically atherosclerosis, remains a leading cause of death in developed countries. Atherosclerosis is a multifactorial disease that results in a narrowing, or stenosis, of a vessel lumen. Briefly, pathologic inflammatory responses resulting from vascular endothelium injury causes monocytes and vascular smooth muscle cells (VSMCs) to migrate from the sub endothelium and into the arterial wall's intimal layer. There the VSMC proliferate and lay down an extracellular matrix causing vascular wall thickening and reduced vessel patency.

Cardiovascular disease caused by stenotic coronary arteries is commonly treated using either coronary artery by-pass graft (CABG) surgery or angioplasty. Angioplasty is a percutaneous procedure wherein a balloon catheter is inserted into the coronary artery and advanced until the vascular stenosis is reached. The balloon is then inflated restoring arterial patency. One angioplasty variation includes arterial stent deployment. Briefly, after arterial patency has been restored, the balloon is deflated and a vascular stent is inserted into the vessel lumen at the stenosis site. The catheter is then removed from the coronary artery and the deployed stent remains implanted to prevent the newly opened artery from constricting spontaneously. However, balloon catheterization and stent deployment can result in vascular injury ultimately leading to VSMC proliferation and neointimal formation within the previously opened artery. This biological process whereby a previously opened artery becomes re-occluded is referred to as restenosis.

The introduction of intracoronary stents into clinical practice has dramatically changed treatment of obstructive coronary artery disease. Since having been shown to significantly reduce restenosis as compared to percutaneous transluminal coronary angioplasty (PTCA) in selected lesions, the indication for stent implantation was been widened substantially. As a result of a dramatic increase in implantation numbers worldwide in less selected and more complex lesions, in-stent restenosis (ISR) has been identified as a new medical problem with significant clinical and socioeconomic implications. The number of ISR cases is growing: from 100,000 patients treated worldwide in 1997 to an estimated 150,000 cases in 2001 in the United States alone. ISR is due to a vascular response to injury, and this response begins with endothelial denudation and culminates in vascular remodeling after a significant phase of smooth muscle cell proliferation.

At least four distinct phases of reaction can be observed in ISR: thrombosis, inflammation, proliferation, and vessel remodeling. There is a wide spectrum of conventional catheter-based techniques for treatment of ISR, ranging from plain balloon angioplasty to various atherectomy devices and repeat stenting. One possible method for preventing restenosis is the administration of anti-inflammatory compounds that block local invasion/activation of monocytes thus preventing the secretion of growth factors that may trigger VSMC proliferation and migration. Other potentially anti-restenotic compounds include anti-proliferative agents such as chemotherapeutics including rapamycin and paclitaxel. However, anti-inflammatory and anti-proliferative compounds can be toxic when administered systemically in anti-restenotic-effective amounts. Furthermore, the exact cellular functions that must be inhibited and the duration of inhibition needed to achieve prolonged vascular patency (greater than six months) is not presently known. Moreover, it is believed that each drug may require its own treatment duration and delivery rate. Therefore, in situ, or site-specific drug delivery using anti-restenotic coated stents has become the focus of intense clinical investigation. Once the coated stent is deployed, it releases the anti-restenotic agent directly into the tissue thus allowing for clinically effective drug concentrations to be achieved locally without subjecting the recipient to side effects associated with systemic drug delivery. Moreover, localized delivery of anti-proliferative drugs directly at the treatment site eliminates the need for specific cell targeting technologies.

Human clinical studies on stent-based anti-restenotic delivery have demonstrated excellent short-term anti-restenotic effectiveness. However, side effects including vascular erosion have also been seen. Vascular erosion can lead to stent instability and further vascular injury. Furthermore, the extent of cellular inhibition may be so extensive that normal re-endothelialization will not occur. The endothelial lining is essential for maintaining vascular elasticity and as an endogenous source of nitric oxide. Therefore, compounds that exert localized anti-restenotic effects while minimizing vascular and cellular damage are essential for the long-term success of drug delivery stents.

### SUMMARY OF THE INVENTION

The present invention is directed at engineering polymers that provide optimized drug eluting medical devices coatings. Specifically, polymers made in accordance with teachings of the present invention provide durable biocompatible coatings for medical devices intended for use in hemodynamic environments. In one embodiment of the present invention vascular stents are provided with a controlled-release polymer coating using the compositions of the present invention. Vascular stents are chosen for exemplary purposes only. Those skilled in the art of material science and medical devices will realize that the polymer compositions of the present invention are useful in coating a large range of medical devices. Therefore, the use of the vascular stent as an exemplary embodiment is not intended as a limitation.

The amphiphilic copolymers of the present invention are useful for coating medical devices with peptide drugs. Therefore, it is an object of the present invention to provide amphiphilic polymers with improved biocompatibility and hemocompatibility for use in drug delivery of peptides via medical devices.

### BRIEF DESCRIPTION OF THE FIGURES

FIG. 1 graphically depicts idealized first-order kinetics associated with drug release from a polymer coating.

FIG. 2 graphically depicts idealized zero-order kinetics associated with drug release from a polymer coating.

FIG. 3 depicts a vascular stent used to deliver the antirestenotic compounds of the present invention.

FIG. 4 depicts a balloon catheter assembly used for angioplasty and the site-specific delivery of stents to anatomical lumens at risk for restenosis.

FIG. 5 depicts the needle of an injection catheter in the retracted position (balloon deflated) according to the principles of the present invention where the shaft is mounted on an intravascular catheter.

### DEFINITION OF TERMS

Prior to setting forth the invention, it may be helpful to an understanding thereof to set forth definitions of certain terms that will be used hereinafter:

Amphiphilic: As used herein “amphiphilic” refers to a molecule having a polar, water-soluble group attached to a nonpolar, water-insoluble hydrocarbon chain.

Animal: As used herein “animal” shall include mammals, fish, reptiles and birds. Mammals include, but are not limited to, primates, including humans, dogs, cats, goats, sheep, rabbits, pigs, horses and cows.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero-order kinetics), that is the drug is released in even amounts over a predetermined time (with or without an initial burst phase) or may be a gradient release. A gradient release implies that the concentration of drug released from the device surface changes over time.

Compatible: As used herein “compatible” refers to a composition possessing the optimum, or near optimum combination of physical, chemical, biological and drug release kinetic properties suitable for a controlled release coating made in accordance with the teachings of the present invention. Physical characteristics include durability and elasticity/ductility, chemical characteristics include solubility and/or miscibility and biological characteristics include biocompatibility. The drug release kinetic should be either near zero-order or a combination of first and zero-order kinetics.

Copolymer: As used here in a “copolymer” will be defined as ordinarily used in the art of polymer chemistry. A copolymer is a macromolecule produced by the simultaneous or step-wise polymerization of two or more dissimilar units such as monomers. Copolymer shall include bipolymer (two dissimilar units) terpolymer (three dissimilar units) etc.

Drug(s): As used herein “drug” shall include any bioactive compound having a therapeutic effect in an animal. Exemplary, non limiting examples include anti-proliferatives including, but not limited to, hydrophilic compounds and peptides such as Angiotensin-(1-7) and biologically active analogues and derivatives thereof.

Ductility: As used herein “ductility, or ductile” is a polymer attribute characterized by the polymer's resistance to fracture or cracking when folded, stressed or strained at operating temperatures. When used in reference to the polymer coating compostions of the present invention the normal operating temperature for the coating will be between room temperature and body temperature or approximately between 15° C. and 40° C. Polymer durability in a defined environment is often a function of its elasticity/ductility.

Glass Transition Point: As used herein “glass transition point” or “Tg” is the temperature at which an amorphous polymer becomes hard and brittle like glass. At temperatures above its Tg a polymer is elastic or rubbery; at temperatures below its Tg the polymer is hard and brittle like glass. Tg may be used as a predictive value for elasticity/ductility.

Hydrophilic: As used herein a hydrophilic molecule or portion of a molecule is one that typically is electrically polarized and capable of hydrogen bonding, enabling it dissolve more readily in water than in oil or other “non-polar” solvents. In reference to bioactive compounds or drugs, the term “hydrophilic” refers to a bioactive compound that has a solubility in water of more than 200 micrograms per milliliter.

Hydrophobic: As used herein a hydrophobic molecule or portion of a molecule is one that typically is electrically neutral and does not hydrogen bond, enabling it dissolve more readily in oil or other “non-polar” solvents rather than in water or “polar” solvents. In reference to bioactive compounds or drugs the term “hydrophobic” refers to a bioactive compound that has a solubility in water of no more than 200 micrograms per milliliter.

Treatment Site: As used herein “treatment site” shall mean a vascular occlusion, vascular plaque, an aneurysm site or other vascular-associated pathology.

### DETAILED DESCRIPTION OF THE INVENTION

The present invention is directed at engineering polymers that provide optimized drug eluting medical devices coatings. Specifically, polymers made in accordance with teachings of the present invention provide durable biocompatible coatings for medical devices intended for use in hemodynamic environments. In one embodiment of the present invention vascular stents are provided with a controlled-release polymer coating using the compositions of the present invention. Vascular stents are chosen for exemplary purposes only. Those skilled in the art of material science and medical devices will realize that the polymer compositions of the present invention are useful in coating a large range of medical devices. Therefore, the use of the vascular stent as an exemplary embodiment is not intended as a limitation.

The amphiphilic copolymers of the present invention are useful for coating medical devices with peptide drugs. Therefore, it is an object of the present invention to provide amphiphilic polymers with improved biocompatibility and hemocompatibility for use in drug delivery of peptides via medical devices.

The amphiphilic copolymers of the present invention are useful for coating medical devices with peptide drugs. Peptides are incompatible with the solvents used in standard hydrophobic polymer coatings. To overcome the solubility issue resulting from the hydrophobic nature of most polymers, a hydrophilic compound such as poly(ethylene glycol) (PEG) can be copolymerized with known biocompatible polymer monomer, such as methacrylate. PEG is probably one of the most well-known hydrophilic polymers and incorporation of PEG in a polymer will increase biocompatibility and hemocompatibility. PEG has additional desirable properties in addition to hydrophilicity and solubility in organic solvents, including an established safety profile and absence of immunogenicity in mammals which allows PEG to be used for many clinical applications. Amphiphilic copolymers containing PEG are used for biomaterials applications because of their unique structure and physical properties. The copolymer of the present invention is composed of PEG-methacrylate and cyclohexyl methacrylate.

Vascular stents present a particularly unique challenge for the medical device coating scientist. Vascular stents (hereinafter referred to as “stents”) must be flexible, expandable, biocompatible and physically stable. Stents are used to relieve the symptoms associated with coronary artery disease caused by occlusion in one or more coronary artery. Occluded coronary arteries result in diminished blood flow to heart muscles causing ischemia induced angina and in severe cases myocardial infarcts and death. Stents are generally deployed using catheters having the stent attached to an inflatable balloon at the catheter's distal end. The catheter is inserted into an artery and guided to the deployment site. In many cases the catheter is inserted into the femoral artery or of the leg or carotid artery and the stent is deployed deep within the coronary vasculature at an occlusion site.

Vulnerable plaque stabilization is another application for coated drug-eluting vascular stents. Vulnerable plaque is composed of a thin fibrous cap covering a liquid-like core composed of an atheromatous gruel. The exact composition of mature atherosclerotic plaques varies considerably and the factors that affect an atherosclerotic plaque's make-up are poorly understood. However, the fibrous cap associated with many atherosclerotic plaques is formed from a connective tissue matrix of smooth muscle cells, types I and III collagen and a single layer of endothelial cells. The atheromatous gruel is composed of blood-borne lipoproteins trapped in the sub-endothelial extracellular space and the breakdown of tissue macrophages filled with low density lipids (LDL) scavenged from the circulating blood. (G. Pasterkamp and E. Falk. 2000. Atherosclerotic Plaque Rupture: An Overview. J. Clin. Basic Cardiol. 3:81-86). The ratio of fibrous cap material to atheromatous gruel determines plaque stability and type. When atherosclerotic plaque is prone to rupture due to instability it is referred to a “vulnerable” plaque. Upon rupture the atheromatous gruel is released into he blood stream and induces a massive thrombogenic response leading to sudden coronary death. Recently, it has been postulated that vulnerable plaque can be stabilized by stenting the plaque. Moreover, vascular stents having a drug-releasing coating composed of matrix metalloproteinase inhibitor dispersed in, or coated with (or both) a polymer may further stabilize the plaque and eventually lead to complete healing.

Treatment of aneurysms is another application for drug-eluting stents. An aneurysm is a bulging or ballooning of a blood vessel usually caused by atherosclerosis. Aneurysms occur most often in the abdominal portion of the aorta. At least 15,000 Americans die each year from ruptured abdominal aneurysms. Back and abdominal pain, both symptoms of an abdominal aortic aneurysm, often do not appear until the aneurysm is about to rupture, a condition that is usually fatal. Stent grafting has recently emerged as an alternative to the standard invasive surgery. A vascular graft containing a stent (stent graft) is placed within the artery at the site of the aneurysm and acts as a barrier between the blood and the weakened wall of the artery, thereby decreasing the pressure on artery. The less invasive approach of stent-grafting aneurysms decreases the morbidity seen with conventional aneurysm repair. Additionally, patients whose multiple medical comorbidities make them excessively high risk for conventional aneurysm repair are candidates for stent-grafting. Stent grafting has also emerged as a new treatment for a related condition, acute blunt aortic injury, where trauma causes damage to the artery.

Once positioned at the treatment site the stent or graft is deployed. Generally, stents are deployed using balloon catheters. The balloon expands the stent gently compressing it against the arterial lumen clearing the vascular occlusion or stabilizing the aneurysm. The catheter is then removed and the stent remains in place permanently. Most patients return to a normal life following a suitable recovery period and have no reoccurrence of coronary artery disease associated with the stented occlusion. However, in some cases the arterial wall's intima is damaged either by the disease process itself or as the result of stent deployment. This injury initiates a complex biological response culminating is vascular smooth muscle cell hyperproliferation and occlusion, or restenosis at the stent site.

Recently significant efforts have been devoted to preventing restenosis. Several techniques including brachytherapy, excimer laser, and pharmacological techniques have been developed. The least invasive and most promising treatment modality is the pharmacological approach. A preferred pharmacological approach involves the site-specific delivery of cytostatic or cytotoxic drugs directly to the stent deployment area. Site-specific delivery is preferred over systemic delivery for several reasons. First, many cytostatic and cytotoxic drugs are highly toxic and cannot be administered systemically at concentrations needed to prevent restenosis. Moreover, the systemic administration of drugs can have unintended side effects at body locations remote from the treatment site. Additionally, many drugs are either not sufficiently soluble, or too quickly cleared from the blood stream to effectively prevent restenosis. Therefore, administration of anti-restenotic compounds directly to the treatment area is preferred.

Several techniques and corresponding devices have been developed to deploy anti-restenotic compounds including weeping balloon and injection catheters. Weeping balloon catheters are used to slowly apply an anti-restenotic composition under pressure through fine pores in an inflatable segment at or near the catheter's distal end. The inflatable segment can be the same used to deploy the stent or a separate segment. Injection catheters administer the anti-restenotic composition by either emitting a pressurized fluid jet, or by directly piercing the artery wall with one or more needle-like appendage. Recently, needle catheters have been developed to inject drugs into an artery's adventitia. However, administration of anti-restenotic compositions using weeping and injection catheters to prevent restenosis remains experimental and largely unsuccessful. Direct anti-restenotic composition administration has several disadvantages. When anti-restenotic compositions are administered directly to the arterial lumen using a weeping catheter, the blood flow quickly flushes the anti-restenotic composition down stream and away from the treatment site. Anti-restenotic compositions injected into the lumen wall or adventitia may rapidly diffuse into the surrounding tissue. Consequently, the anti-restenotic composition may not be present at the treatment site in sufficient concentrations to prevent restenosis. As a result of these and other disadvantages associated with catheter-based local drug delivery, investigators continue to seek improved methods for the localized delivery of anti-restenotic compositions.

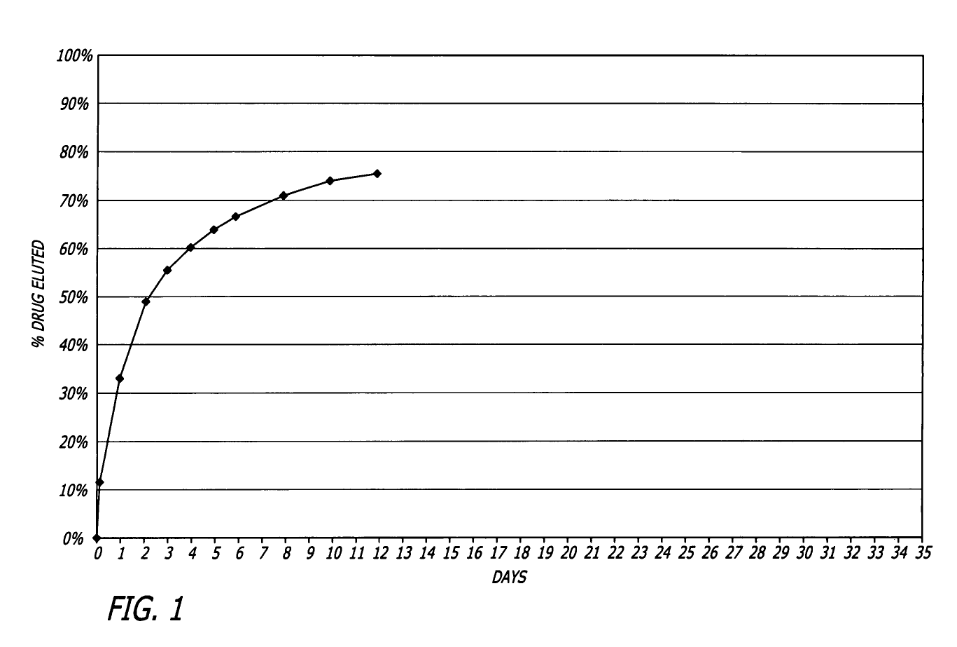

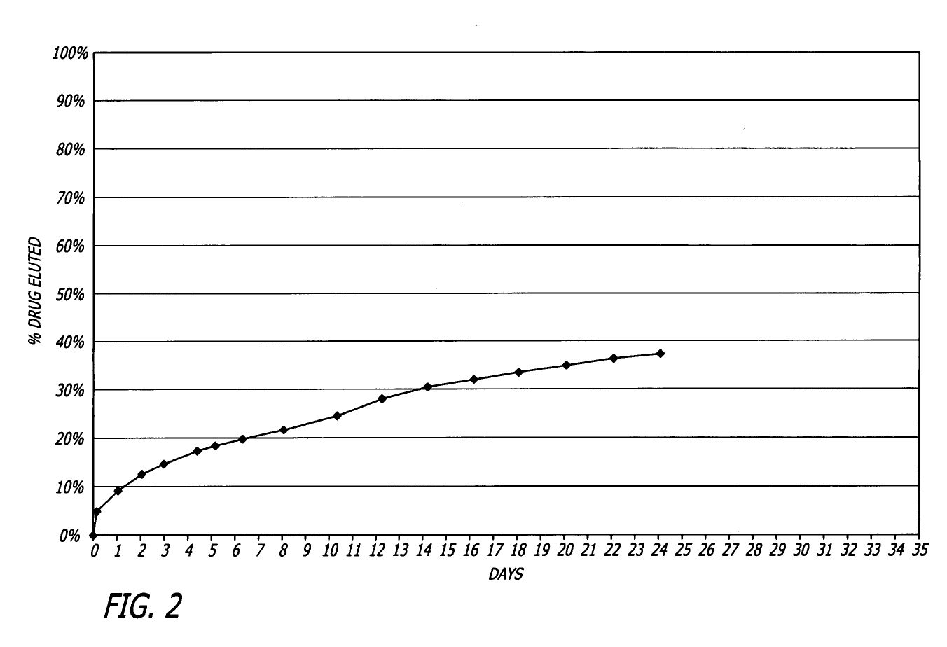

The most successful method for localized anti-restenotic composition delivery developed to date is the drug-eluting stent. Many-drug eluting stent embodiments have been developed and tested. However, significant advances are still necessary in order to provide safe and highly effective drug delivery stents. One of the major challenges associated with stent-based anti-restenotic composition delivery is controlling the drug delivery rate. Generally speaking, drug delivery rates have two primary kinetic profiles. Drugs that reach the blood stream or tissue immediately after administration follow first-order kinetics. FIG. 1 graphically depicts idealized first-order kinetics. First-order drug release kinetics provide an immediate surge in blood or local tissue drug levels (peak levels) followed by a gradual decline (trough levels). In most cases therapeutic levels are only maintained for a few hours. Drugs released slowly over a sustained time where blood or tissue concentrations remains steady follow zero-order kinetics. FIG. 2 graphically depicts idealized zero-order kinetics. Depending on the method of drug delivery and tissue/blood clearance rates, zero-order kinetics result in sustained therapeutic levels for prolonged periods. Drug-release profiles can be modified to meet specific applications. Generally, most controlled release compositions are designed to provide near zero-order kinetics. However, there may be applications where an initial burst, or loading dose, of drug is desired (first-order kinetics) followed by a more gradual sustained drug release (near zero-order kinetics).

The present invention is directed at optimized drug releasing medical device coatings suitable for use in hemodynamic environments. The coatings of the present invention are composed of polymers having at least one bioactive compound or drug dispersed therein. The polymeric compositions of the present invention have been specifically formulated to provide medical device coatings that tenaciously adhere to medical device surfaces (do not delaminate), flex without fracturing (ductile), resist erosion (durable), are biocompatible and release a wide variety of drugs at controlled rates.

Polymers have been used as medical device coatings for decades to enhance biocompatibility and erosion resistance. Moreover, in certain applications polymer coatings may also provide electrical insulation. It is also well known in the art that polymers can act as reservoirs and/or diffusion barriers to control biological agent elution rates.

Recently, coatings have been applied to implantable medical devices such as vascular stents, vascular stent grafts, urethral stents, bile duct stents, catheters, inflation catheters, injection catheters, guide wires, pace maker leads, ventricular assist devices, and prosthetic heart valves. Devices such as these are generally subjected to flexion strain and stress during implantation, application or both. Providing flexible medical devices such as stents with stable biocompatible polymer coatings is especially difficult.

There are two basic molecular morphologies that define a polymer's tertiary solid-state structure. Polymers may be either semi-crystalline or amorphous depending on the nature of the polymer subunit. Semi-crystalline polymers are rigid and brittle at any temperature below their melting point and are generally not suitable for coating flexible medical devices such as stents. In addition, drugs or bioactive compounds cannot stay in the polymer crystal region, therefore, the drug or bioactive agent loading is limited. Amorphous polymers, on other hand, can be either rigid or elastic/ductile depending on its glass transition point (Tg). The Tg of an amorphous polymer is the temperature above which the amorphous polymer is elastic/ductile and flexible. For stent application it is desirable that the Tg be below body temperature. Many polymeric compostions have Tgs substantially above body temperature and are thus in the glassy or rigid state when the device is deployed and remains so once the device is implanted. Polymers in the “glassy” state are non-elastic/ductile and prone to cracking, fracturing and delaminating when the stent is flexed. Polymer coatings susceptible to fracture and delaminating are especially undesirable when used on stents. Small polymer particles that separate from a delaminated or fractured stent coating may be carried by the blood flow downstream where they can lodge in capillaries and obstruct blood flow to critical regions of the heart. Therefore stents and other flexible medical devices should have polymer coatings that are elastic/ductile and adhere to the device surface well. Generally, this requires that coating polymers be amorphous and have glass transition points below body temperature.

However, polymers having extremely low Tgs are undesirable when used to coat devices that are subjected to continual hemodynamic forces. As general rule, the lower the Tg the more rubbery a polymer backbone becomes. More rubbery polymers can be tacky. This is partially due to the fact that the more rubbery polymers have higher coefficient of friction. Therefore, polymers having extremely low Tgs should not be the dominant polymer in polymer blends or copolymer compostions when designing coating polymers intend for stents and other vascular implants. In addition, extremely low Tg (e.g., rubbery) polymers tend to release drugs or bioactive materials at undesirably fast rates due to their high free volumes.

In addition to the aforementioned structural and drug releasing profile considerations, polymers used as stent coatings must also be biocompatible. Biocompatibility encompasses numerous factors that have been briefly defined in the preceding “Definition of Terms” section. The need for a polymer to be biocompatible significantly limits the number of available options for the material scientist. Moreover, these options are further limited when the polymer coating is used on a device that is continuously exposed to hemodynamic forces. For example, stent coatings must remain non-thrombogenic, non-inflammatory and structurally stable for prolonged time periods.

There are generally two large, and to some extent overlapping, categories of biocompatible polymers suitable as medical device coatings: bioerodable (including bioresorbable polymers) and non-bioerodable polymers. Coating compositions of the present invention are principally directed at the latter. The remaining discussion and exemplary embodiments will be directed at non-bioerodable polymers.

Non-erodable polymers can be hydrophilic, hydrophobic or amphiphilic depending on the polarity of the monomers used and the ratio of hydrophobic to hydrophilic monomers. Hydrophilic polymers are polar molecules that are miscible with polar solvents and are generally lubricious while contacting body fluids and are often used in biomedical applications to produce lubricious hydrogels. However, hydrogel polymers can be unstable in a hemodynamic environment and lack physical integrity because of their high water content. Moreover, many hydrophobic drugs do not disperse well in hydrogels and therefore hydrogels are not suitable drug delivery platforms for some hydrophobic bioactive compounds. Hydrophobic polymers are nonpolar molecules that are soluble in nonpolar solvents. There are biocompatible hydrophobic polymers; however, may of these have a high coefficient of frictions which is undesirable in a hemodynamic environment. Moreover, many hydrophilic drugs do not disperse well in hydrophobic polymers and therefore are not suitable drug delivery platforms for many hydrophilic bioactive compounds.

Therefore, there are four specific attributes that the stent coating polymers made in accordance with the teachings of the present invention should possess. The polymer compositions of the present invention should be biocompatible, durable, elastic/ductile and possess a predetermined drug release profile. Other requirements include processing compatibility such as inert to sterilization methods including, but not limited to, ethylene oxide sterilization. The present invention provides novel polymer compositions made in accordance with the teachings of the present invention.

Release rate is not entirely a function of drug-polymer compatibility. Coating configurations, polymer swellability and coating thickness also play roles. When the medical device of the present invention is used in the vasculature, the coating dimensions are generally measured in micrometers (μm). Coatings consistent with the teaching of the present invention may be a thin as 1 μm or a thick as 1000 μm. There are at least two distinct coating configurations within the scope of the present invention. In one embodiment of the present invention the drug-containing coating is applied directly to the device surface or onto a polymer primer. Depending on the solubility rate and profile desired, the drug is either entirely soluble within the polymer matrix, or evenly dispersed throughout. The drug concentration present in the polymer matrix ranges from 0.1% by weight to 80% by weight. In either event, it is most desirable to have as homogenous of a coating composition as possible. This particular configuration is commonly referred to as a drug-polymer matrix.

Finally, returning to coating thickness, while thickness is generally a minor factor in determining overall drug-release rates and profile, it is nevertheless an additional factor that can be used to tune the coatings. Basically, if all other physical and chemical factors remain unchanged, the rate at which a given drug diffuses through a given coating is directly proportional to the coating thickness. That is, increasing the coating thickness increases the elution rate and visa versa.

We now turn to another factor that contributes to the compatiblized controlled release coatings of the present invention. As mentioned earlier, coating intended for medical devices deployed in a hemodynamic environment must possess excellent adhesive properties. That is, the coating must be stably linked to the medical device surface. Many different materials can be used to fabricate the implantable medical devices including stainless steel, nitinol, aluminum, chromium, titanium, ceramics, and a wide range of synthetic polymeric and natural materials including collagen, fibrin and plant fibers. All of these materials, and others, may be used with the controlled release coatings made in accordance with the teachings of the present invention.

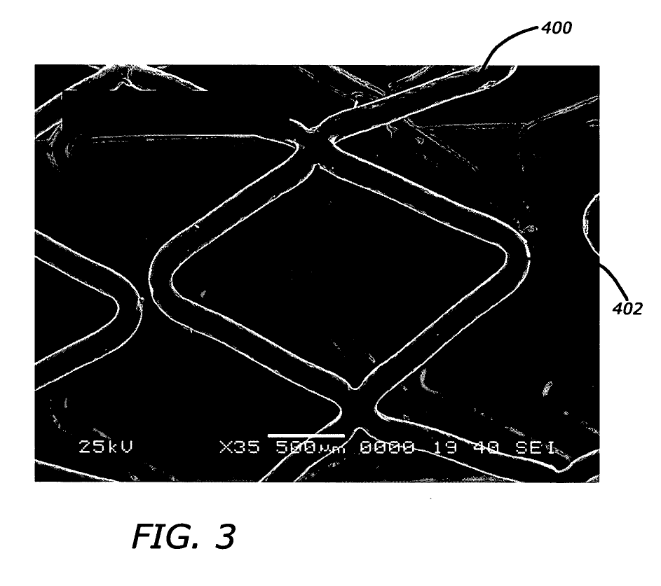

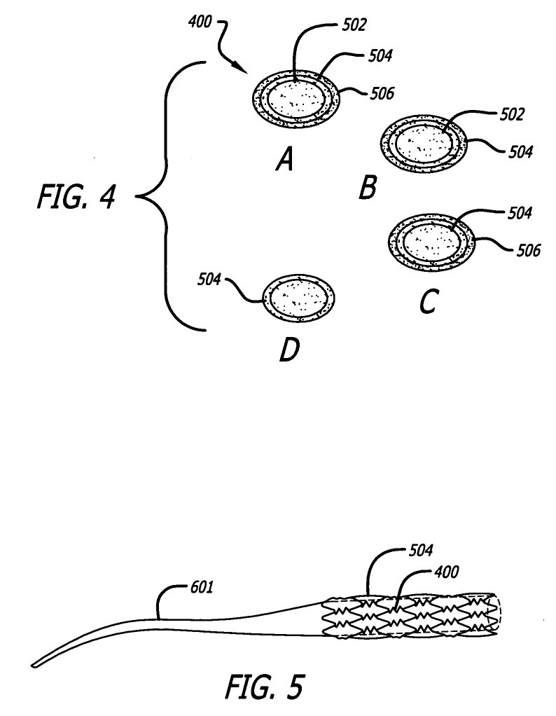

One embodiment of the present invention is depicted in FIG. 3. In FIG. 3 a vascular stent 400 having the structure 402 is made from a material selected from the non-limiting group materials including stainless steel, nitinol, aluminum, chromium, titanium, ceramics, and a wide range of synthetic polymeric and natural materials including collagen, fibrin and plant fibers. The structure 402 is provided with a coating composition made in accordance with the teachings of the present invention. FIG. 4a-d are cross-sections of stent 400 showing various coating configurations. In FIG. 4a stent 400 has a first polymer coating 402 comprising an optional medical grade primer, such as but not limited to parylene; a second controlled release coating 404; and a third barrier, or cap, coat 406. In FIG. 4b stent 400 has a first polymer coating 402 comprising an optional medical grade primer, such as but not limited to parylene and a second controlled release coating 404. In FIG. 4c stent 400 has a first controlled release coating 404 and a second barrier, or cap, coat 406. In FIG. 4d stent 400 has only a controlled release coating 404. FIG. 5 depicts a vascular stent 400 having a coating 504 made in accordance with the teachings of the present invention mounted on a balloon catheter 501.

There are many theories that attempt to explain, or contribute to our understanding of how polymers adhere to surfaces. The most important forces include electrostatic and hydrogen bonding. However, other factors including wettability, absorption and resiliency also determine how well a polymer will adhere to different surfaces. Therefore, polymer base coats, or primers are often used in order to create a more uniform coating surface.

The controlled release coatings of the present invention can be applied to medical device surfaces, either primed or bare, in any manner known to those skilled in the art. Applications methods compatible with the present invention include, but are not limited to, spraying, dipping, brushing, vacuum-deposition, and others. Moreover, the controlled release coatings of the present invention may be used with a cap coat. A cap coat as used here refers to the outermost coating layer applied over another coating. A drug-releasing copolymer coating is applied over the primer coat. A polymer cap coat is applied over the drug-releasing copolymer coating. The cap coat may optionally serve as a diffusion barrier to further control the drug release, or provide a separate drug. The cap coat may be merely a biocompatible polymer applied to the surface of the sent to protect the stent and have no effect on elution rates.

Most polymers used for stent coating applications are hydrophobic and as such are not soluble in solvents compatible for use with peptides. Suitable polymers for releasing peptides are amphiphilic polymers. To overcome the solubility issue resulting from the hydrophobic nature of most polymers, a hydrophilic compound such as poly(ethylene glycol) (PEG) can be copolymerized with a known biocompatible polymer monomer, such as a methacrylate. PEG is probably one of the most well-known hydrophilic polymers and incorporation of PEG in a polymer will increase biocompatibility and hemocompatibility. PEG has additional desirable properties in addition to hydrophilicity and solubility in organic solvents, including an established safety profile and absence of immunogenicity in mammals which allows PEG to be used for many clinical applications. Amphiphilic copolymers containing PEG are used for biomaterials applications because of their unique structure and physical properties. The copolymer of the present invention comprises PEG-methacrylate and cyclohexyl methacrylate.

The copolymers of the present invention have the general structure of Formula 1 wherein a, b and n are independently integers from 1-100 and n is the length of the PEG tail; R₁ is H or lower alkyl and R₂ is H, substituted or unsubstituted C₁-C₁₀₀ straight or branched chain alkyl, alkenyl, cycloalkyl, or cycloalkenyl groups, substituted or unsubstituted phenyl or benzyl group, heterocyclic groups, multi-cyclic alkyl or alkenyl groups, including, without limitation norbornyl and adamantyl groups. Substituent groups may include, but are not limited to halogens, hydroxyl groups, carboxyl groups, alkoxy groups, oxygen, nitrogen, sulfur, phosphorous, gallium, iron, boron and one or more radioisotope of same.

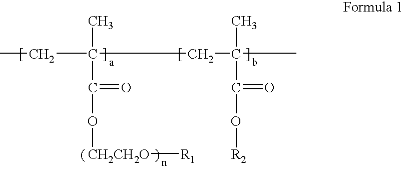

One embodiment of the present invention is a copolymer of PEG-methacrylate and cyclohexyl methacrylate designated C45.

The examples are meant to illustrate one or more embodiments of the invention and are not meant to limit the invention to that which is described below.

### EXAMPLE 1

### Synthetic Methods for C45 Copolymer

Synthetic Scheme 1

6.0 g of poly(ethylene glycol) methyl ether methacrylate (Sigma-Aldrich, molecular weight 300), 4.0 g of cyclohexyl methacrylate (Sigma-Aldrich), 20 mL toluene and 37 mg of 2,2′-azobisisobutyronitrile (AIBN) were mixed in a bottle with a magnetic stirring bar. The bottle was sealed and purged with N₂ for 20 minutes. The reaction bottle was heated for 2 hours while stirring in a water bath kept at 60° C. The polymer was precipitated in hexanes five times. A polymer with number average molecular weight (Mn)=217,000 daltons and weight average molecular weight (Mw)=632,000 daltons was obtained after drying under vacuum.

Synthetic Scheme 2

6.0 g of poly(ethylene glycol) methacrylate (Sigma-Aldrich, molecular weight 360), 4.0 g of cyclohexyl methacrylate (Sigma-Aldrich), 28 mL of acetone, 12 mL of 1-butanol and 37 mg of AIBN were mixed in a bottle with a magnetic stirring bar. The bottle was sealed and purged with N₂ for 20 minutes. The reaction bottle was heated for 77 minutes while stirring in a water bath kept at 60° C. The polymer was precipitated in hexanes two times and in water three times. A polymer with Mn=149,000 daltons and Mw=1,126,000 daltons was obtained after drying under vacuum.

Synthetic Scheme 3

5.0 g of poly(ethylene glycol) methacrylate (Sigma-Aldrich, molecular weight 360), 5.0 g of ethyl methacrylate (Sigma-Aldrich), 14 mL of acetone, 6 mL of 1-butanol and 38 mg of AIBN were mixed in a bottle with a magnetic stirring bar. The bottle was sealed and purged with N₂ for 20 minutes. The reaction bottle was heated in a water bath kept at 60° C. for 3 hours while stirring. The polymer was precipitated in hexanes three times and in water three times. A polymer with Mn=176,000 daltons and Mw=969,000 daltons was obtained after drying under vacuum.

What is claimed is:

1. A medical device for providing the controlled release of an anti-restenotic drug comprising a vascular stent coated with an amphiphilic copolymer and an anti-restenotic drug.

2. An amphiphilic copolymer for providing controlled release coatings on medical devices comprising hydrophilic and hydrophobic polymers of the formula: 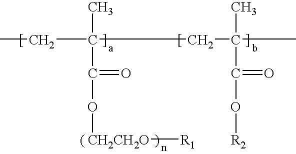
wherein a, b and n are integers from 1-100; R₁ is H or lower alkyl and R₂ is H, a substituted or unsubstituted C₁-C₁₀₀ straight or branched chain alkyl, alkenyl, cycloalkyl, or cycloalkenyl group, a substituted or unsubstituted phenyl or benzyl group, heterocyclic groups, a multi-cyclic alkyl or alkenyl group.

3. The amphiphilic copolymer according to claim 2 wherein R₁ comprises a C₁₋₁₀₀alkyl, a C₁₋₁₀₀ alkenyl, or H.

4. The amphiphilic copolymer according to claim 2 wherein R₂ is selected from the group consisting, methyl, ethyl, cyclohexyl, norbornyl, phenyl, benzyl and adamantyl.

5. The amphiphilic copolymer according to claim 2 wherein said substituent groups are selected from the group consisting of halogens, hydroxyl groups, carboxyl groups, alkoxy groups, oxygen, nitrogen, sulfur, phosphorous, gallium, iron, boron and one or more radioisotope of same.

6. A drug-containing controlled releasing coating for a medical device comprising the amphiphilic copolymer of claim 2 and a drug.

7. The drug-releasing coating of claim 6 wherein the amphiphilic copolymer is a poly(ethylene glycol) (PEG)-methacrylate-cyclohexyl methacrylate copolymer.

8. The drug-releasing coating of claim 6 wherein said drug is an anti-restenotic drug.

9. The drug-releasing coating of claim 8 wherein said drug is a peptide.

10. The drug-releasing coating of claim 6 wherein said medical device is a vascular stent.

11. A medical device for providing the controlled release of an anti-restenotic composition comprising:
a stent having a generally cylindrical shape comprising an outer surface, an inner surface, a first open end and a second open end and wherein at least one of said inner or outer surfaces are adapted to deliver an anti-restenotic effective amount of at least one drug to a tissue within a mammal.

12. The medical device according to claim 11 wherein said stent is mechanically expandable.

13. The medical device according to claim 11 wherein said stent is self expandable.

14. The medical device according to claim 11 wherein at least one anti-restenotic drug is present on both said inner surface and said outer surface of said stent.

15. The medical device according to claim 11 wherein at least one of said inner and outer surfaces are coated with an amphiphilic copolymer wherein said amphiphilic copolymer has a least one anti-restenotic drug incorporated therein and said amphiphilic copolymer releases said at least one anti-restenotic drug into said tissue of said mammal.

16. The medical device according to claim 11 wherein said stent is delivered to said tissue using a balloon catheter.

17. The medical device according to claim 11 wherein said tissue is an anatomical lumen.

18. The medical device according to claim 17 wherein said tissue is a blood vessel lumen.

19. A vascular stent coated with an amphiphilic copolymer coating containing an anti-restenotic amount of a anti-restenotic drug.

20. The vascular stent of claim 19 further comprising a parylene primer coat.

21. The vascular stent of claim 19 wherein said amphiphilic copolymer coating comprises a PEG methacrylate-cyclohexyl methacrylate polymer.

22. The vascular stent of claim 19 further comprising a poly(butyl) methacrylate topcoat.

23. The vascular stent of claim 19 wherein said anti-restenotic drug is a peptide.

24. The vascular stent of claim 23 wherein said peptide is in a concentration of between approximately 0.1% to 99% by weight of peptide-to-polymer.

25. The vascular stent according to claim 19 wherein said stent is delivered to a tissue of a mammal's anatomical lumen using a balloon catheter.

26. A method of inhibiting restenosis in a mammal comprising the site specific delivery of at least one anti-restenotic drug.

27. The method according to claim 26 wherein said anti-restenotic drug is delivered to a site at risk for restenosis using a vascular stent.

28. The method according to claim 26 wherein said anti-restenotic drug is delivered to a site at risk for restenosis using an injection catheter.

29. A method for inhibiting restenosis comprising providing a vascular stent having a controlled release coating comprising an amphiphilic copolymer and an anti-restenotic effective amount of an anti-restenotic drug
